(19) United States
(12) Patent Application Publication
(10) Pub. No.: US 2017/0246046 A1
(43) Pub. Date: Aug. 31, 2017

(54) DISPOSABLE ARTICLE WITH FOLDED HANDLE AND METHOD OF FORMING

(71) Applicant: Kimberly-Clark Worldwide, Inc., Neenah, WI (US)

(72) Inventors: Julie Ann Paveletzke, Appleton, WI (US); Andrew Mark Long, Appleton, WI (US); Joseph Daniel Coenen, Neenah, WI (US); Bradley Adam Colvin, Appleton, WI (US); Joseph Charles Herb, Greenville, WI (US); Adam Edward Suttner, Appleton, WI (US); Christopher Peter Olson, Neenah, WI (US)

(21) Appl. No.: 15/514,035

(22) PCT Filed: Oct. 28, 2015
(86) PCT No.: PCT/US15/57725

§ 371 Date: Mar. 24, 2017

Related U.S. Application Data
(60) Provisional application No. 62/073,374, filed on Oct. 31, 2014.

(51) Int. Cl. A61F 13/49

(52) U.S. Cl. CPC A61F 13/49; A61F 2013/49098

(57) ABSTRACT

Pant-like absorbent articles having handles on side panels of the article for ease of removal and donning of the absorbent garment and a method for forming is disclosed herein. The article includes a front waist region, a back waist region, and a chassis extending longitudinally between and interconnecting the front and back waist regions attached by at least one side panel. Formed within the side panel is at least one handle. The handle includes a slit formed through the side panel, the slit extending in both the longitudinal and lateral direction to provide a flap material. The flap material is folded towards the waist edge of the side panel to provide an aperture in the side panel.

### RELATED APPLICATIONS

The present application claims priority to U.S. Provisional Application No. 62/073,374, filed Oct. 31, 2014, the contents of which are hereby incorporated by reference in a manner consistent with the present application.

### BACKGROUND

Many absorbent articles intended for personal wear such as diapers, training pants, feminine hygiene products, adult incontinence products, bandages, medical garments and the like are designed to be sufficiently absorbent to pull moisture from liquid body exudates including urine, menses, blood, etc. away from the wearer to reduce skin irritation caused by prolonged wetness exposure. Diapers, as an example, are typically placed and secured on a wearer using a set of primary fastening tabs, such as adhesive tabs or mechanical (e.g., hook or loop) fastening system tabs and left in place to absorb insults as well as to contain fecal waste.

Training pants, unlike diapers, typically come pre-assembled in a wear configuration to more closely resemble conventional underpants. In particular, front and back waist regions of such training pants are typically attached at a seam either permanently or refastenably (such as by a primary fastening system) to define a wear configuration of the pants having a waist opening and leg openings. Such design enables pull-on prefastened garments allowing for self-application and removal.

Unfortunately, current pull-on prefastened garments have not been optimized for child application. While children of potty training age desire to complete tasks independently, they typically lack the dexterity and cognitive ability to successfully don current pull-on prefastened garments on their own. As a result, most of the time the caregiver will either don the product completely, assist the child during the donning process, or readjust the pant after the child tries unsuccessfully on his or her own. In addition to being difficult to grab, there are no cues that direct the child as to where to hold the garment. Left on their own, children typically try to pull on the product by holding the front or the back of the garment. When the garment is pulled on by holding the front, it usually gets stuck beneath the buttocks. Holding the product at the back is difficult for the child and results in the product not coming up completely at the front. In both instances, readjustment is required by the caregiver.

Thus, there is a need to provide an optimized handle for use with absorbent articles that have the necessary strength and design that enables a toddler to successfully pull-on the garment.

### SUMMARY

The present disclosure is directed to pant-like absorbent articles having handles on side panels of the article for ease of removal and donning of the absorbent garment. In one embodiment, the article includes a liquid permeable inner surface for facing the wearer, an outer surface for facing away from the wearer, an absorbent body disposed therebetween. The article further includes a front waist region, a back waist region, and a crotch region extending longitudinally between and interconnecting the front and back waist regions. At least one side panel is attached to the front waist region and the back waist region to define a wear configuration of the absorbent article having a waist opening and a leg opening spaced from the waist opening, wherein the at least one side panel extends from the waist opening to the leg opening. Formed within the side panel is at least one handle. The handle includes a slit formed through the side panel, the slit extending in both the longitudinal and lateral direction to provide a flap material, the flap material folded towards the waist edge of the side panel to provide an aperture in the side panel.

In another embodiment, a method for forming an absorbent article for personal wear about a wearer's waist is disclosed. At least one side panel material is provided and a slit is formed through the side panel. The slit extends in both the longitudinal and lateral direction to provide a foldable slit material. The folded slit material is folded towards a waist edge of the side panel to provide an aperture in the side panel. An absorbent assembly comprising a liquid permeable inner surface for facing the wearer, an outer surface for facing away from the wearer, an absorbent body disposed therebetween, and a front waist region, a back waist region, and a crotch region extending longitudinally between and interconnecting the front and back waist regions is provided. Then the at least one side panel is attached to the front waist region and the back waist region to define a wear configuration of the absorbent article having a waist opening and a leg opening spaced from the waist opening, wherein the at least one side panel extends from the waist opening to the leg opening.

### DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a bottom plan view of one aspect of a personal wear article in the form of a pair of training pants having an handle as described herein, in an unfastened, unfolded and laid flat condition, and showing the surface of the training pants that faces away from the wearer;

FIG. 2 is a top plan view of the product of FIG. 1 showing the surface of the training pants that faces the wearer when worn and with portions cut away to show underlying features;

FIG. 3 is a partial schematic view of the handle illustrated in FIGS. 1-2;

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DEFINITIONS

It is to be understood by one of ordinary skill in the art that the present discussion is a description of exemplary aspects of the present disclosure only, and is not intended as limiting the broader aspects of the present disclosure.

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Connected” refers to the joining, adhering, bonding, attaching, or the like, of two elements. Two elements will be considered to be connected together when they are connected directly to one another or indirectly to one another, such as when each is directly connected to intermediate elements.

“Cross direction” refers to the width of a fabric in a direction generally perpendicular to the direction in which it is produced, as opposed to “machine direction” that refers to the length of a fabric in the direction in which it is produced.

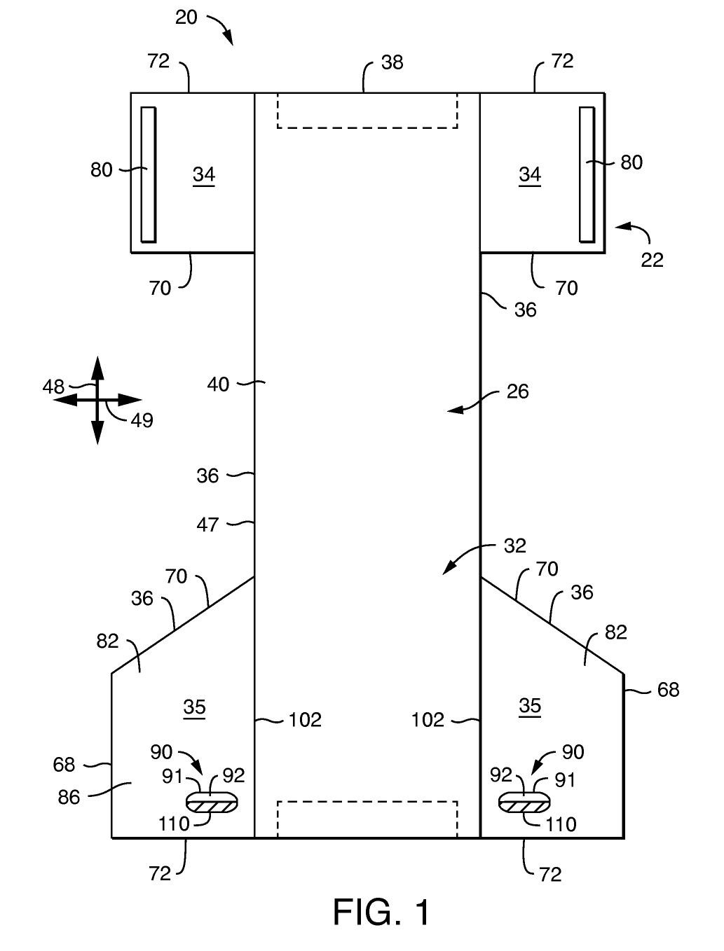

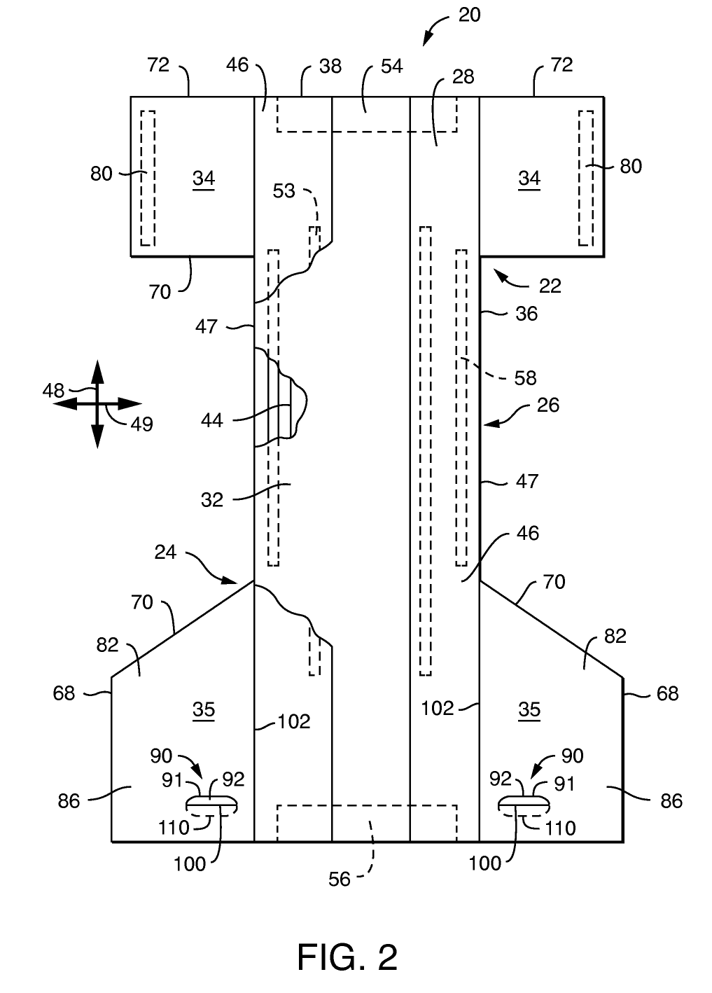

“Cross direction assembly” refers to a process in which disposable absorbent products are manufactured in an orientation in which the products are connected side-to-side, in the transverse direction shown by arrow 49 in FIGS. 1 and 2, a process utilizing a cross direction assembly that entails products traveling through a converting machine parallel to the direction of arrow 49, as opposed to “machine direction assembly” in which the products are connected end-to-end or waist-to-waist.

“Disposable” refers to articles that are designed to be discarded after a limited use rather than being laundered or otherwise restored for reuse.

“Disposed,” “disposed on,” and variations thereof are intended to mean that one element can be integral with another element, or that one element can be a separate structure bonded to or placed with or placed near another element.

“Elastic,” “elasticized” and “elasticity” mean that property of a material or composite by virtue of which it tends to recover its original size and shape after removal of a force causing a deformation.

“Elastomeric” refers to a material or composite that can be elongated by at least 25 percent of its relaxed length and that will recover, upon release of the applied force, at least 10 percent of its elongation. It is generally preferred that the elastomeric material or composite be capable of being elongated by at least 100 percent, more preferably by at least 300 percent, of its relaxed length and recover, upon release of an applied force, at least 50 percent of its elongation.

“Fabrics” is used to refer to any woven, knitted and nonwoven fibrous webs.

“Film” refers to a thermoplastic film made using a film extrusion and/or forming process, such as a cast film or blown film extrusion process. The term includes apertured films, slit films, and other porous films that constitute liquid transfer films, as well as films that do not transfer liquid.

“Flexible” refers to materials that are compliant and that will readily conform to the general shape and contours of the wearer's body.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

“Integral” or “integrally” is used to refer to various portions of a single unitary element rather than separate structures bonded to or placed with or placed near one another.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, can spread or be transported parallel to the plane of the liquid impermeable layer or laminate, but this is not considered to be within the meaning of “liquid impermeable” when used herein.

“Liquid permeable material” or “liquid water-permeable material” refers to a material present in one or more layers, such as a film, nonwoven fabric, or open-celled foam, which is porous, and which is water permeable due to the flow of water and other aqueous liquids through the pores. The pores in the film or foam, or spaces between fibers or filaments in a nonwoven web, are large enough and frequent enough to permit leakage and flow of liquid water through the material.

“Longitudinal” and “transverse” have their customary meaning, as indicated by the longitudinal and transverse axes depicted in FIGS. 1 and 2. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Machine direction” refers to the length of a fabric in the direction in which it is produced, as opposed to “cross direction” that refers to the width of a fabric in a direction generally perpendicular to the machine direction.

“Machine direction assembly” refers to a process in which disposable absorbent products are manufactured in an orientation in which the products are connected end-to-end or waist-to-waist, in the longitudinal direction shown by arrow 48 in FIGS. 1 and 2, a process utilizing a machine direction assembly entails products traveling through a converting machine parallel to the direction of arrow 48, as opposed to “cross direction assembly” in which the products are connected side-to-side.

“Meltblown fiber” means fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers that can be continuous or discontinuous, are generally smaller than about 0.6 denier, and are generally self bonding when deposited onto a collecting surface. Meltblown fibers used in the present disclosure are preferably substantially continuous in length.

“Member” when used in the singular can have the dual meaning of a single element or a plurality of elements.

“Nonwoven” and “nonwoven web” refer to materials and webs of material that are formed without the aid of a textile weaving or knitting process.

“Operatively joined,” in reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Permanently bonded” refers to the joining, adhering, connecting, attaching, or the like, of two elements of an absorbent garment such that the elements tend to be and remain bonded during normal use conditions of the absorbent garment.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Refastenable” refers to the property of two elements being capable of releasable attachment, separation, and subsequent releasable reattachment without substantial permanent deformation or rupture.

“Releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment. It should be noted that a releasably attached or releasably engaged seam is a refastenable seam that does not include a bonded seam that must be torn, cut, or otherwise disrupted.

“Spunbonded fiber” refers to small diameter fibers that are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Stretchable” means that a material can be stretched, without breaking, to at least 150% of its initial (unstretched) length in at least one direction, suitably to at least 200% of its initial length, desirably to at least 250% of its initial length.

“Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

“Thermoplastic” describes a material that softens when exposed to heat and that substantially returns to a nonsoftened condition when cooled to room temperature.

These terms can be defined with additional language in the remaining portions of the specification.

### DETAILED DESCRIPTION

Referring now to the drawings and in particular to FIGS. 1 and 2, a personal wear absorbent article according to one aspect is illustrated in the form of a pants-type article for wear about a wearer's waist, and more particularly in the form of children's toilet training pants, indicated in its entirety by the reference numeral 20. The term absorbent generally refers to articles that can be placed against or in proximity to the body of the wearer to absorb and/or retain various liquid wastes discharged from the body. The absorbent article can be disposable, which refers to articles that are intended to be discarded after a limited period of use instead of being laundered or otherwise restored for reuse. It is understood that the concepts described herein are suitable for use with various other pants-type articles such as adult incontinence articles, as well as other articles intended for personal wear such as clothing, diapers, feminine hygiene products, medical garments, surgical pads and bandages, other personal care or health care garments, and the like without departing from the scope of the present disclosure.

By way of illustration only, various materials and methods for constructing the training pants 20 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., which are incorporated herein by reference.

The present disclosure is directed to a pant-like absorbent garment having handles on side panels of the garment for ease of removal and donning of the absorbent garment. The handle of the refastenable side seam training pants is designed with strength sufficient to allow a user of the product to pull on the garment without tearing the product. When a toddler or caregiver grasps a handle on the side panel to pull-on the product, the toddler or caregiver must apply enough force to pull the product on.

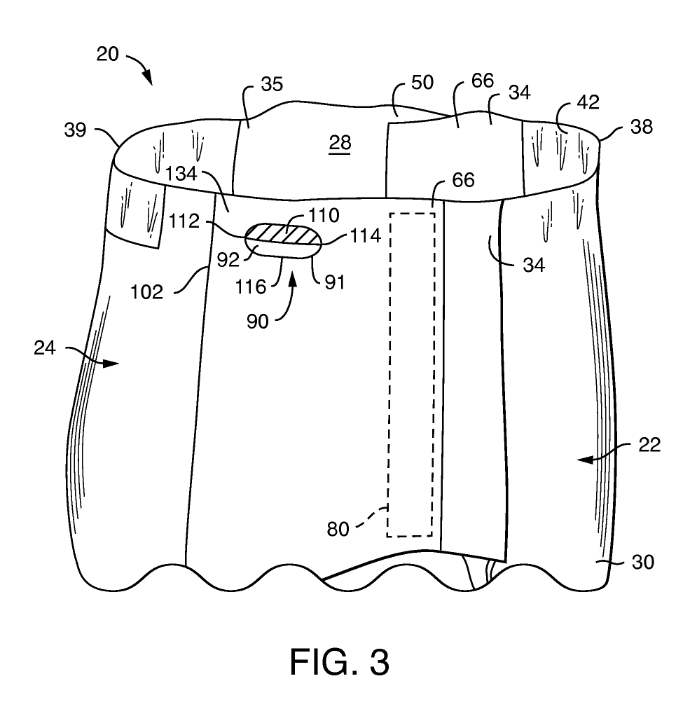

The pair of training pants 20 is illustrated in FIG. 3 in a fully pre-assembled (i.e., as assembled during initial manufacture) configuration (broadly referred to herein as a wear configuration of the pants, i.e., absorbent article). With additional reference to FIGS. 1 and 2, the training pants 20 includes a front waist region 22, a back waist region 24, a crotch region 26 extending longitudinally between and interconnecting the front and back waist regions 22, 24 along a longitudinal direction of the pants, an inner surface 28 configured for contiguous relationship with the wearer, and an outer surface 30 opposite the inner surface 28. The training pants 20 also has a pair of laterally opposite side edges 36 and a pair of longitudinally opposite waist edges, respectively designated front waist edge 38 and back waist edge 39. The front waist region 22 is contiguous with the front waist edge 38, and the back waist region 24 is contiguous with the back waist edge 39.

The illustrated pants 20 includes a central absorbent assembly, generally indicated at 32, which when laid flat as in FIGS. 1 and 2 can be rectangular or any other desired shape. A pair of laterally opposite front side panels 34 extends outward from the absorbent assembly 32 at the front waist region 22 (thereby forming transversely outer portions of the front waist region 22, and more broadly in part forming transversely opposite sides of the training pants). Laterally opposite back side panels 35 extend outward from the absorbent assembly 32 at the back waist region 24 (thereby forming transversely outer portions of the back waist region 24, and together with the front side panels 34 further defining the sides of the pants).

The central absorbent assembly 32 of the illustrated aspect includes an outer cover 40 and a bodyside liner 42 (FIG. 3) connected to the outer cover 40 in a superposed relation by suitable means such as adhesives, ultrasonic bonds, thermal bonds or other conventional techniques. An absorbent structure 44 (FIG. 2) is disposed between the outer cover 40 and the bodyside liner 42. A pair of containment flaps 46 (FIG. 2) is secured to the bodyside liner 42 for inhibiting the lateral flow of body exudates. The central absorbent assembly 32 has opposite ends that form portions of the front and back waist edges 38 and 39, and opposite side edges 47 that form portions of the side edges 36 of the training pants 20 (FIGS. 1 and 2).

The absorbent assembly 32 and side panels 34, 35 can include two or more separate elements, as shown in FIGS. 1 and 2, or they can be integrally formed. Integrally formed side panels 34, 35 and absorbent assembly 32 would include at least some common materials, such as the bodyside liner 42, flap composite, outer cover 40, other materials and/or combinations thereof, and could define one-piece elastic, stretchable, or nonstretchable pants 20. For further reference, arrows 48 and 49 in FIGS. 1 and 2 depict the orientation of a longitudinal axis and a transverse or lateral axis, respectively, of the training pants 20.

The front waist region 22 of the training pant 20 can be selectively joined to the back waist region 24 via a pair of refastenable side seams 66 via an article fastening system 80 to define a pull-on, pant-like configuration of the training pant having a waist opening, indicated at 50, and two leg openings. The article fastening system 80 may include any suitable complementary refastenable fasteners including, for example and without limitation, hook- and loop-type fasteners, other types of mechanical fasteners, adhesive fasteners, cohesive fasteners, and combinations thereof. In some suitable embodiments, the fastening system 80 may be pre-fastened during the manufacturing process of the training pant 20 such that the training pant 20 is supplied to the user in a fastened configuration. While FIG. 3 illustrates the front and back regions 22, 24 being joined together via refastenable seams 66, it is understood that the front and back regions can be joined together via non-refastenable, bonded seams (e.g., by adhesive bonding, ultrasonic bonding, pressure bonding, thermal bonding).

With the training pants 20 in the fastened condition as illustrated in FIG. 3, the front and back side panels 34, 35 are attached to each other by a primary, or article fastening system 80 to define the pre-assembled three-dimensional wear configuration of the pants 20, having a waist opening 50 and a pair of leg openings. The front waist region 22 includes the portion of the training pants 20 that, when worn, is positioned at least in part on the front of the wearer while the back waist region 24 includes the portion of the training pants 20 that is positioned at least in part on the back of the wearer. The crotch region 26 of the training pants 20 includes the portion of the training pants 20 that is positioned between the legs of the wearer and covers the lower torso of the wearer.

The front and back side panels 34 and 35 include the portions of the training pants 20 (and more particularly of the front and back waist regions 22, 24) that, when worn, are positioned on the hips of the wearer. The attached side panels 34, 35 thus broadly define the transversely opposite sides of the pants 20 at a refastenable seam 66 along which the fastening system 80 releasably attaches the front and back side panels 34, 35. The waist edges 38 and 39 of the training pants 20 are configured to encircle the waist of the wearer and together define the waist opening 50 (FIG. 3). Portions of the side edges 36 in the crotch region 26 generally define leg openings. The side panels 34 and 35 desirably have a panel length dimension measured parallel to the longitudinal axis 48 along the outer edge 68. Optionally, the panel length dimension of the back side panel 35 is longer than the front side panel 34.

The central absorbent assembly 32 is configured to contain and/or absorb exudates discharged from the wearer. For example, the containment flaps 46 are configured to provide a barrier to the transverse flow of body exudates. A flap elastic member 53 (FIG. 2) can be operatively joined with each containment flap 46 in any suitable manner as is well known in the art. The elasticized containment flaps 46 define a partially unattached edge that assumes an upright configuration in at least the crotch region 26 of the training pants 20 to form a seal against the wearer's body. The containment flaps 46 can be located along the side edges 36 of the pants 20, and can extend longitudinally along the entire length of the absorbent assembly 32 or can only extend partially along the length of the absorbent assembly 32. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 also suitably includes a front waist elastic member 54 (FIG. 2), a rear waist elastic member 56, and leg elastic members 58, as are known to those skilled in the art. The waist elastic members 54 and 56 can be attached to the outer cover 40 and/or the bodyside liner 42 along the opposite waist edges 38 and 39, and can extend over part or all of the waist edges. The leg elastic members 58 can be attached to the outer cover 40 and/or the bodyside liner 42 along the opposite side edges 36 and positioned in the crotch region 26 of the training pants 20. The leg elastic members 58 can be longitudinally aligned along each side edge 47 of the absorbent assembly 32.

The outer cover 40 suitably includes a material that is substantially liquid impermeable. The outer cover 40 can be a single layer of liquid impermeable material, but more suitably includes a multi-layered laminate structure in which at least one of the layers is liquid impermeable. The inner layer of the outer cover 40 can be both liquid and vapor impermeable, or it can be liquid impermeable and vapor permeable.

It is also contemplated that the outer cover 40 can be stretchable, and more suitably elastic. In particular, the outer cover 40 is suitably stretchable and more suitably elastic in at least the transverse or circumferential direction of the pants 20. In other aspects the outer cover 40 can be stretchable, and more suitably elastic, in both the transverse and the longitudinal direction.

The liquid permeable bodyside liner 42 is illustrated as overlying the outer cover 40 and absorbent structure 44, and can, but need not, have the same dimensions as the outer cover 40. The bodyside liner 42 is suitably compliant, soft feeling, and non-irritating to the child's skin. Further, the bodyside liner 42 can be less hydrophilic than the absorbent structure 44 to present a relatively dry surface to the wearer and permit liquid to readily penetrate through its thickness.

Alternatively, the bodyside liner 42 can be more hydrophilic or can have essentially the same affinity for moisture as the absorbent structure 44 to present a relatively wet surface to the wearer to increase the sensation of being wet. This wet sensation can be useful as a training aid. The hydrophilic/hydrophobic properties can be varied across the length, width and/or depth of the bodyside liner 42 and absorbent structure 44 to achieve the desired wetness sensation or leakage performance.

The bodyside liner 42 can also be stretchable, and more suitably elastic. In particular, the bodyside liner 42 is suitably stretchable and more suitably elastic in at least the transverse 49, or circumferential direction of the pants 20. In other aspects, the bodyside liner 42 can be stretchable, and more suitably elastic, in both the transverse 49 and the longitudinal 48 directions.

As noted previously, the illustrated training pants 20 have front and back side panels 34 and 35 defining transversely opposite sides of the pants 20 in the wear configuration of the pants 20. The side panels 34, 35 can be permanently attached along seams 102 to the central absorbent assembly 32 in the respective front and back waist regions 22 and 24. More particularly, as seen best in FIG. 1, the front side panels 34 can be permanently attached to and extend transversely outward beyond the side edges 47 of the absorbent assembly 32 in the front waist region 22, and the back side panels 35 can be permanently attached to and extend transversely outward beyond the side edges 47 of the absorbent assembly 32 in the back waist region 24. The side panels 34 and 35 can be attached to the absorbent assembly 32 using attachment means known to those skilled in the art such as adhesive, thermal, pressure or ultrasonic bonding. Alternatively, the side panels 34 and 35 can be formed as an integral portion of a component of the absorbent assembly 32. For example, the side panels 34, 35 can include a generally wider portion of the outer cover 40, the bodyside liner 42, and/or another component of the absorbent assembly 32.

The front and back side panels 34, 35 each have an outer edge 68 spaced laterally from the seam 66, a leg end edge 70 disposed toward the longitudinal center of the training pants 20, and a waist end edge 72 disposed toward a longitudinal end of the training pants 20. The leg end edge 70 and waist end edge 72 extend from the side edges 47 of the absorbent assembly 32 to the outer edges 68. The leg end edges 70 of the side panels 34 and 35 form part of the side edges 36 of the training pants 20. The leg end edges 70 of the illustrated aspect are suitably curved and/or angled relative to the transverse axis 49 to provide a better fit around the wearer's legs. However, it is understood that only one or both of the leg end edges 70 can be curved or angled, such as the leg end edge 70 of the back waist region 24, or neither of the leg end edges 70 can be curved or angled, without departing from the scope of this disclosure. The waist end edges 72 are suitably parallel to the transverse axis 49. The waist end edges 72 of the front side panels 34 form part of the front waist edge 38 of the training pants 20, and the waist end edges 72 of the back side panels 35 form part of the back waist edge 39 of the pants 20.

The side panels 34, 35 suitably, although not necessarily, include a stretchable material capable of stretching in a direction generally parallel to the transverse axis 49 of the training pants 20. More suitably the side panels 34, 35 include an elastic material. Suitable elastic materials, as well as one process of incorporating stretchable side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. Optionally, the stretch material can include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and PCT application WO 01/88245 in the name of Welch et al.; all of which are incorporated herein by reference. Other suitable materials are described in U.S. patent application Ser. No. 12/649,508 to Welch et al. and U.S. Pat. No. 8,287,677 to Lake et al., all of which are incorporated herein by reference.

Alternatively, the side panel material can include other woven or nonwoven materials, such as those described above as being suitable for the outer cover 40 or bodyside liner 42; mechanically pre-strained composites; or stretchable but inelastic materials.

A handle 90 is formed in at least one side panel of the training pants 20. The handle 90 must have sufficient strength to enable the user to pull on the training pants 20 without causing too much displacement in the material. If the handle 90 is too weak, the material will stretch too far causing the article to be stuck around the knees or buttocks while being pulled on or not being placed in the correct position such that it is snug against the wearer's body and is improperly positioned to accept waste.

The handle 90 is produced in the side panel 35 of a pant 20 using a die cutter or other means such as water-cutter, laser, or the like to form a slit 91. This slit 91 is shaped so it can form a flap material 110 which is folded toward the waist band edge 72 on the garment facing (outside) of the side panel 35. Optionally, this flap material 110 is affixed or bonded in place by a suitable bonding method in a folded position.

The slit 91 may be formed in the at least one side panel 35 by any means known to one skilled in the art. For example, a conventional dividing or severing device, such as a rotary knife, a die cutter, a water cutter, a laser or other energy beam cutter, a high-energy particle-beam cutter or the like, may be employed to form a slit 91 in the side panel material 35.

Optionally, the slit 91 extends in both the longitudinal and lateral direction on the side panel 35 to provide a flap material 110 that may be folded. Desirably, an undulated, u-shaped or v-shaped slit 91 is formed in the side panel 35, forming a flap material 110 and an aperture 92 when the flap material 110 is folded. Desirably, the slit 91 extends laterally having a first end 112, a second end 114, and a middle portion 116. In the embodiment, the first end 112 and the second end 114 are closer longitudinally than the middle portion 116 to the waist edge 72 of the side panel 35.

In some embodiments, the portion of the slit 91 closest to the leg edge 70 of the side panel 35 releases from the cutting mechanism after the portion of the slit 91 closest to the waist edge of the side panel. This causes the cutting mechanism to lift the flap material 110 away from the side panel 35 as the cutting mechanism lifts off of the side panel material, thereby starting the folding of the flap material 110 toward the waist edge 72 of the side panel 35.

This lifting of the flap material 110 may be enabled by varying the structure of the die along the cutting edge causing a late release of the material in the middle portion 116 of the slit 91. For example, the die can be less sharp at the middle portion 116 of the slit 91, causing a late release because it is not cleanly cut in this area or due to having a tight radius near the bottom that does not release the flap material 110 as readily as the top portions of the die.

The lifted flap material 110 is caught and folded by any suitable device for folding the flap material 110 into the desired folded position, including but not limited to folding boards, folding skis, paddles, fingers, vacuum devices, air blasts, mechanical devices with reciprocating motion such as tuckers, four-bar linkages, slide-crank mechanisms, or the like and combinations thereof.

Optionally, the flap material 110 is affixed or bonded in a folded position to the side panel material 35. The flap material 110 may be affixed to the side panel material 35 by any suitable means such as adhesives, ultrasonic bonds, thermal bonds, cohesive bonds, or the like and combinations thereof as known by those skilled in the art.

The flap material 110 being operatively joined to the side panel 35 provides the necessary strength in the product for handle functionality. When the handle 90 is being used to pull-on the product, lateral and longitudinal forces are placed on the handle 90. Operatively joining the flap material 110 to the side panel helps provide the ability to manage greater maximum stress/strain without inhibiting stretch of the handle 90. This provides good balance of displacement and strength. One potential benefit of controlling the force concentrations is to insure they do not occur adjacent to the distal ends of refastenable seams and potentially cause the seam to pop open during donning.

Once the flap material 110 is folded against the side panel 35, an aperture 92 is formed in the side panel 35. A user of the product is then able to grasp the handle 90 through the aperture 92 to remove or put on the pant 20.

The size and location of the handle 90 may also increase the functionality of the handle as it is optimized to enable independence for a toddler who is learning potty training. The maximum width of the aperture 92 must enable a user, typically a toddler child, the ability to place their fingers and hands through the aperture and pull on the product. However, if the maximum width of the aperture 92 is too great, the handle 90 will allow for too much displacement and cause issues with the article's effectiveness. Unexpectedly, it has been found that a toddler will typically place one or two fingers through the aperture 92 and not all four fingers. As a result, an optimal maximum width for the aperture 92 has been found. In one embodiment, the aperture 92 may have a maximum width of between about 20 mm and about 45 mm. Desirably, the aperture 92 has a maximum width of about 30 mm.

The location of the handle 90 can also be important to its ability to function correctly. It has been found that a handle 90 longitudinally close to the waist edge 72 causes excessive displacement/tearing, and longitudinally close to the leg edge 70 is difficult to grab or guide the training pants 20 on the body. As a result, the handle 90 may also have a top edge 100 of the aperture 92 located between about 10 and about 35 mm from the waist edge 72 of the side panel 35. Desirably, a top edge 100 of the aperture 92 located about 20 mm from the waist edge 72 of the side panel 35.

The location of the handle 90 laterally on the side panel 35 may also be considered to improve effectiveness. It has been found that a handle laterally close to the refastenable seam causes excessive displacement/tearing, and laterally close to the effective edge of the side panel is difficult to grab or guide the article on the body. As a result, the handle 90 may also have a back distal side edge of the aperture located between about 10 and about 25 mm from an effective edge of the side panel. Desirably, a back distal side edge of the aperture may be located about 20 mm from the waist edge. For purposes herein and illustrated in FIG. 3, an effective edge 102 of the side panel is the location where the side panel 35 is bonded to the absorbent assembly 32. In cases, where the side panel is integral with the absorbent assembly 32, the effective edge is where the absorbent core is attached to the outer cover 40.

The handle 90 may be formed on the side panel 35 at an angle relative to the lateral axis 49 of the side panel 35 to increase ease of use and strength. Angled handles can enable controlling and directing stress and strain resulting from donning. One potential benefit of controlling the force concentrations is to insure they do not occur adjacent to the distal ends of refastenable seams and potentially cause the seam to pop open during donning. The handle 90 may be placed on the side panel 35 at an angle relative to the lateral axis 49 of the side panel 35 to increase ease of use and strength. Desirably, the handle 90 may be placed at an angle of at least 5 degrees, and more desirably at an angle of between about 10 and about 20 degrees relative to the lateral axis 48 of the side panel 35.

Optionally, the flap material 110 is a different color or pattern than the side panel 35 material. This is a visual cue directing the hand to the handle 90. In addition, the double thickness provided by the folded flap material 110 on the side panel 35 provides tactile cues that help locate the proper hand position for grasping the handle 90. This may make the handle more intuitive for grasping by a toddler.

When introducing elements of the present disclosure or the preferred aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. An absorbent article for personal wear about a wearer's waist, the article having a transverse axis and a longitudinal centerline, the article comprising:
a liquid permeable inner surface for facing the wearer;
an outer surface for facing away from the wearer;
an absorbent body disposed therebetween;
a front waist region, a back waist region, and a crotch region extending longitudinally between and interconnecting the front and back waist regions;
at least one side panel attached to the front waist region and the back waist region to define a wear configuration of the absorbent article having a waist opening and a leg opening spaced from the waist opening, wherein the at least one side panel extends from the waist opening to the leg opening; and
at least one handle comprising a slit formed through the side panel, the slit extending in both the longitudinal and lateral direction to provide a flap material, the flap material folded towards a waist edge of the side panel to provide an aperture in the side panel.

2. The article of claim 1 further comprising the folded slit material bonded in a folded position to the side panel.

3. The article of claim 1 wherein the slit extends laterally having a first end, a second end, and a middle portion, wherein the first end and the second end are closer longitudinally to the waist edge of the waist edge than the middle portion.

4. The article of claim 1 wherein the slit comprises a generally u-shaped or v-shaped slit.

5. The article of claim 1 wherein the slit comprises an undulated shape.

6. The article of claim 1 wherein the side panel further comprises a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening.

7. The article of claim 5 wherein the back side panel is longer than the front side panel.

8. The article of claim 1 wherein the maximum width of the handle is about 30 mm.

9. The article of claim 1 wherein a back distal side edge of the aperture is located between about 10 and about 25 mm from an effective edge of the side panel.

10. The article of claim 1 wherein the folded flap material is a different color or pattern than the side panel material.

11. A method of forming an absorbent article for personal wear about a wearer's waist, the article having a transverse axis and a longitudinal centerline the method comprising:
providing at least one side panel material;
forming a slit through the side panel, the slit extending in both the longitudinal and lateral direction to provide a foldable slit material;
folding the foldable slit material towards a waist edge of the side panel to provide an aperture in the side panel;
providing an absorbent assembly, the absorbent assembly comprising a liquid permeable inner surface for facing the wearer, an outer surface for facing away from the wearer, an absorbent body disposed therebetween, and a front waist region, a back waist region, and a crotch region extending longitudinally between and interconnecting the front and back waist regions; and
attaching the at least one side panel to the front waist region and the back waist region to define a wear configuration of the absorbent article having a waist opening and a leg opening spaced from the waist opening, wherein the at least one side panel extends from the waist opening to the leg opening.

12. The method of claim 11 further comprising bonding the folded slit material to the side panel in a folded position.

13. The method of claim 11 wherein the slit extends laterally having a first end, a second end, and a middle portion, wherein the first end and the second end are closer longitudinally to the waist edge than the middle portion.

14. The method of claim 11 wherein the slit comprises a generally u-shaped or v-shaped slit.

15. The method of claim 11 wherein the slit comprises an undulated shape.

16. The method of claim 11 wherein the side panel further comprises a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening.

17. The method of claim 16 wherein the back side panel is longer than the front side panel.

18. The method of claim 11 wherein the maximum width of the handle is about 30 mm.

19. The method of claim 11 wherein a back distal side edge of the aperture is located between about 10 and about 25 mm from an effective edge of the side panel.

20. The method of claim 11 wherein the foldable flap material is a different color or pattern than the side panel material.
